(19) United States
(12) Patent Application Publication
(10) Pub. No.: US 2007/0098592 A1
(43) Pub. Date: May 3, 2007

(54) Parallel flow VHP decontamination system

(75) Inventors: Peter J. Buczynski, Girard, PA (US); Francis J. Zelina, Lake City, PA (US); Aaron L. Hill, Erie, PA (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 11/263,796

(22) Filed: Nov. 1, 2005

(51) Int. Cl. A61L 2/24; A61L 2/20

(52) U.S. Cl. 422/3; 422/292; 422/305; 422/30

(57) ABSTRACT

A closed loop vapor decontamination system for decontaminating a defined region. A chamber defines the region. A first fluid flow path connects at both ends to the chamber to define a closed loop path through the chamber. A second fluid flow path connects at both ends to the chamber to define a closed loop path through the chamber. The system has a means for conveying a carrier gas simultaneously along the first and second fluid flow paths. A generator generates vaporized hydrogen peroxide and is disposed along the first fluid flow path for introducing vaporized hydrogen peroxide into the carrier gas as it circulates through the first fluid flow path. A destroyer converts the vaporized hydrogen peroxide into water and oxygen and is disposed along the second fluid flow path for breaking down the vaporized hydrogen peroxide in the carrier gas as it circulates through the second fluid flow path. A controller operates to control the amount of the carrier gas flowing along the first and second fluid flow paths.

### FIELD OF THE INVENTION

The present invention relates generally to the art of sterilization and decontamination, and more particularly to a system for controlling the humidity level in a sterilization or decontamination system that uses a sterilant in its gaseous or vapor phase.

### BACKGROUND OF THE INVENTION

Gaseous and vapor sterilization/decontamination systems rely on maintaining certain process parameters in order to achieve a target sterility or decontamination assurance level. For Vaporized Hydrogen Peroxide (VHP) sterilization/decontamination systems, one such critical process parameter is the humidity level within the space where sterilization is to occur. By controlling the humidity level, it is possible to reduce the condensation of the hydrogen peroxide due to vapor saturation and thereby provide a more efficient sterilization/decontamination cycle. Conventional Vaporized Hydrogen Peroxide (VHP) sterilization systems for decontaminating large rooms or isolators are generally closed loop systems that include both a vaporizer and a dryer in the same flow path. When the dryer and the vaporizer are in the same flow path, the amount of air flow through the system is determined by balancing the needs of the dryer versus the vaporizer. A problem with such systems is that the humidity level in a large room or isolator space to be sterilized cannot be easily controlled during a sterilization/decontamination cycle.

The present invention overcomes this and other problems, and provides a decontamination system that allows for varying humidity levels during a sterilization/decontamination cycle, independent of the flow through the vaporizer.

### SUMMARY OF THE INVENTION

In accordance with the present invention, there is provided a closed loop vapor decontamination system for decontaminating a defined region. The system has a chamber that defines a region. A first fluid flow path is connected at both ends to the chamber to define a first closed loop path through the chamber. A second fluid flow path is connected at both ends to the chamber to define a second closed loop path through the chamber. Means for conveying a carrier gas simultaneously along the first and second fluid flow paths. A generator for generating vaporized hydrogen peroxide is disposed along the first fluid flow path for introducing vaporized hydrogen peroxide into the carrier gas as it circulates through the first fluid flow path. A destroyer for converting the vaporized hydrogen peroxide into water and oxygen, is disposed along the second fluid flow path for breaking down the vaporized hydrogen peroxide in the carrier gas as it circulates through the second fluid flow path. A controller controls the amount of the carrier gas flowing along the first and second fluid flow paths.

In accordance with another aspect of the present invention, there is provided a method for controlling the humidity level in an isolator or room, comprising the steps of: providing a sealable region, a first fluid flow path and a second fluid flow path, said first fluid flow path and said second fluid flow path both include said sealable region; conveying a flow of a carrier gas simultaneously along said first fluid flow path and said second fluid flow path; introducing vaporized hydrogen peroxide into said carrier gas flowing along said first fluid flow path; and destroying said vaporized hydrogen peroxide in said carrier gas flowing along said second fluid flow path.

In accordance with another aspect of the present invention, there is provided another method for controlling the humidity level in an isolator or room, comprising the steps of: providing a decontamination system having a sealable region, a first fluid flow path and a second fluid flow path that both include said sealable region and a sensor disposed within said sealable region operable to monitor the conditions within said sealable region to provide signals indicative of said conditions; conveying a flow of a carrier gas simultaneously along said first fluid flow path and said second fluid flow path; introducing vaporized hydrogen peroxide into said carrier gas flowing along said first fluid flow path; destroying said vaporized hydrogen peroxide in said carrier gas flowing along said second fluid flow path; and varying the relative flow of carrier gas along said first fluid flow path and said second fluid flow path based on signals from said sensor.

An advantage of the present invention is a system for the sterilization or decontamination of a large room or isolated space.

Another advantage of the present invention is a system, as described above, that allows for varying the humidity level in the room or isolator during the operating cycle.

Another advantage of the present invention is a system, as described above, that reduces the pressure drop throughout the entire system to provide for more efficient flow of the carrier gas.

Another advantage of the present invention is a system, as described above, that provides independent flow through a vaporizer and a dryer to allow for smaller blowers to be utilized.

This and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a schematic view of a vaporized hydrogen peroxide deactivation system illustrating a preferred embodiment of the present invention;

FIG. 2 is a schematic drawing of a control system for the vaporized hydrogen peroxide decontamination system shown in FIG. 1;

FIG. 3 is a schematic view of a vaporized hydrogen peroxide deactivation system illustrating an alternative embodiment of the present invention with independent first and second fluid flow paths;

FIG. 4 is a schematic drawing of a control system for the vaporized hydrogen peroxide decontamination system shown in FIG. 3;

FIG. 5. is a schematic view of a vaporized hydrogen peroxide deactivation system illustrating another alternative embodiment of the present invention with first, second and third fluid flow paths;

FIG. 6 is a schematic drawing of a control system for the vaporized hydrogen peroxide decontamination system shown in FIG. 5;

FIG. 7 is a schematic view of a vapor hydrogen peroxide deactivation system illustrating another alternative embodiment of the present invention with independent first and second fluid flow paths and a bypass conduit around the dryer in the second fluid flow path; and

FIG. 8 is a schematic drawing of a control system for the vaporized hydrogen peroxide decontamination system shown in FIG. 7.

### DETAILED DESCRIPTION OF PREFERRED EMBODIMENT

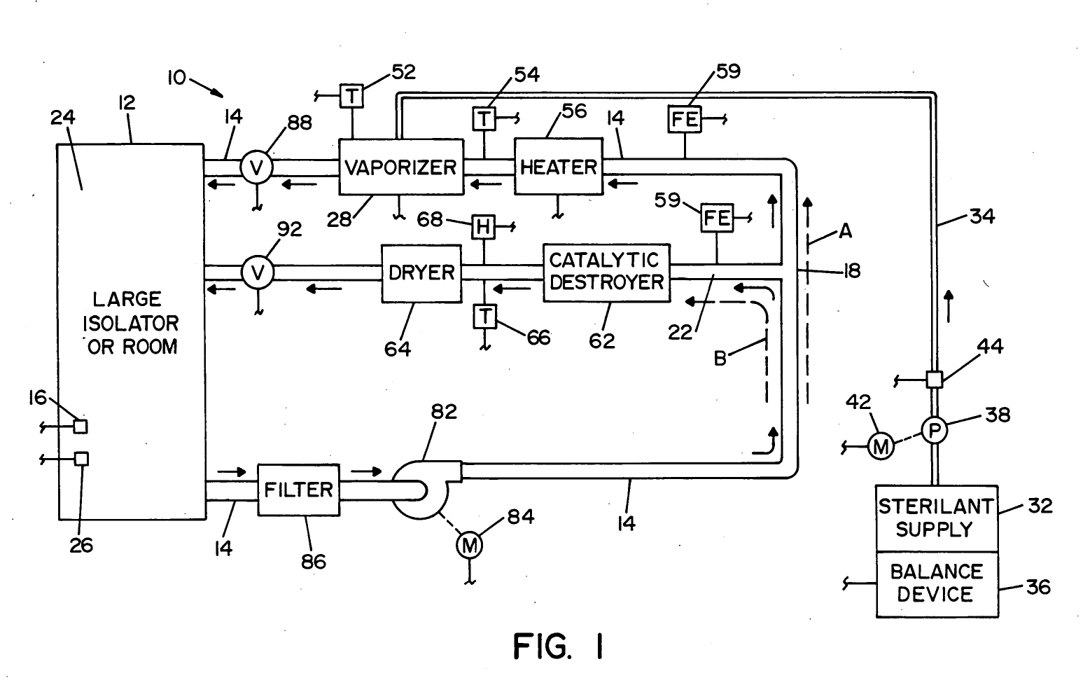

Referring now to the drawings wherein the showings are for the purpose of illustrating a preferred embodiment of the invention only, and not for the purpose of limiting same, FIG. 1 shows a vaporized hydrogen peroxide sterilization system 10, illustrating a preferred embodiment of the present invention. System 10 includes an isolator or room 12 that defines an inner sterilization/decontamination chamber or region 24. Articles to be sterilized or decontaminated may be disposed within isolator or room 12. A first humidity sensor 16 is disposed within isolator or room 12. First humidity sensor 16 is operable to provide a variable electrical signal that is proportional to the humidity of the carrier gas within isolator or room 12. A VHP sensor 26 is disposed within isolator or room 12. VHP sensor 26 can be an electrochemical cell that gives a signal proportional to the gas concentration or it can be a near infrared spectrophotometer that provides a similar signal or some other commercially available sensor for detecting the concentration of VHP in an isolator or room.

System 10 is comprised of a first fluid flow path “A” and a second fluid flow path “B.” First fluid flow path “A” is defined by isolator or room 12 and a first conduit 14. One end of first conduit 14 connects to isolator or room 12. The other end of first conduit 14 also connects to isolator or room 12. In this respect, isolator or room 12 and first conduit 14 define a closed loop path. Second fluid flow path “B” is defined by isolator or room 12, a portion of first conduit 14 and a second conduit 22. One end of second conduit 22 connects to first conduit 14 at a junction 18. The other end of second conduit 22 connects to isolator or room 12. In this respect, isolator or room 12, a portion of first conduit 14 and second conduit 22 defined a closed loop path.

A vaporizer 28 (also referred to herein as generator) is disposed along first fluid flow path “A” to introduce vaporized hydrogen peroxide into first fluid flow path “A.” Vaporizer 28 is connected to a liquid sterilant supply 32 by a feed line 34. A conventionally known balance device 36 is associated with sterilant supply 32, to measure the actual mass of sterilant being supplied to vaporizer 28. A pump 38 driven by a motor 42 is provided to convey metered amounts of the liquid sterilant to vaporizer 28 where the sterilant is vaporized by conventionally known means. In an alternate embodiment, pump 38 is provided with an encoder (not shown) that allows monitoring of the amount of sterilant being metered to vaporizer 28. If an encoder is provided with pump 38, balance device 36 is not required. A pressure switch 44 is provided in the feed line. Pressure switch 44 is operable to provide an electrical signal in the event that a certain static head pressure does not exist in feed line 34.

A VHP temperature sensor 52 is disposed on vaporizer 28 to measure the temperature of the VHP exiting vaporizer 28. VHP temperature sensor 52 is operable to provide a variable electrical signal that is proportional to the temperature of the VHP exiting vaporizer 28. A vaporizer inlet temperature sensor 54 is provided to measure the temperature of the carrier gas entering vaporizer 28. Vaporizer inlet temperature sensor 54 is operable to provide a variable electrical signal that is proportional to the temperature of the carrier gas entering vaporizer 28. A heater 56 is provided prior to vaporizer 28. Heater 56 is operable to heat the carrier gas circulating through first fluid flow path “A.” In this respect, the carrier gas is heated prior to the carrier gas entering vaporizer 28. A first flow element 59 provides a variable electrical signal that is proportional to the air flow entering vaporizer 28. A second flow element 59 provides a variable electrical signal that is proportional to the air flow entering destroyer 62.

A destroyer 62 is disposed along second fluid flow path “B” to destroy hydrogen peroxide (H₂O₂) flowing therethrough, as is conventionally known. Catalytic destroyer 62 converts the hydrogen peroxide (H₂O₂) into water and oxygen. A dryer 64 is disposed along second fluid flow path “B.” Dryer 64 is located downstream of destroyer 62. In this respect, dryer 64 is disposed between destroyer 62 and chamber or region 24. Dryer 64 is operable to remove moisture from the carrier gas flowing through second fluid flow path “B.” A catalytic destroyer temperature sensor 66 and a second humidity sensor 68 are disposed along second fluid flow path “B.” Catalytic destroyer temperature sensor 66 and second humidity sensor 68 are located upstream of dryer 64, as seen in FIG. 1. Catalytic destroyer temperature sensor 66 is operable to provide a variable electrical signal that is proportional to the temperature of the carrier gas exiting catalytic destroyer 62. Second humidity sensor 68 is operable to provide a variable electrical signal that is proportional to the humidity of the carrier gas exiting catalytic destroyer 62.

A blower 82, driven by a motor 84, is provided to circulate a carrier gas simultaneously along first fluid flow path “A” and second fluid flow path “B.” A filter 86 is provided upstream of blower 82. Filter 86 is operable to filter dirt and/or debris from the carrier gas circulated along first fluid flow path “A” and second fluid flow path “B.” A first valve 88 is provided to regulate flow along first conduit 14. First valve 88 is a variable flow valve. A second valve 92 is provided to regulate flow along second conduit 22. Second valve 92 is a variable flow valve.

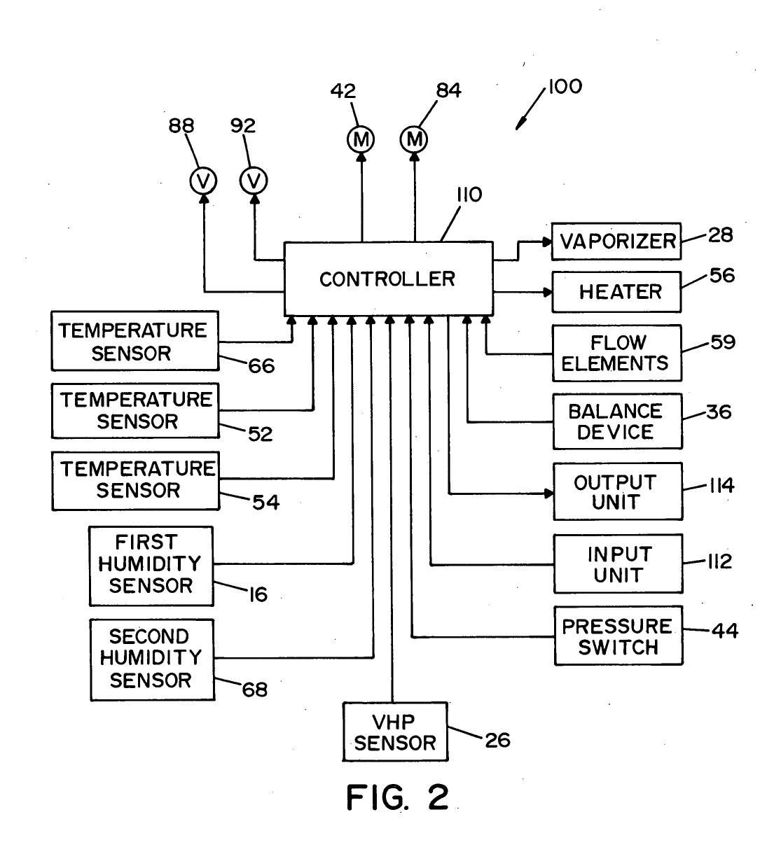

Referring now to FIG. 2, a control system 100 for controlling the operation of system 10 is schematically illustrated. Control system 100 includes a controller 110 that controls the operations of motors 42, 84 and valves 88 and 92. Controller 110 also monitors VHP sensor 26, pressure switch 44, VHP temperature sensor 52, vaporizer inlet temperature sensor 54, catalytic destroyer temperature sensor 66, balance device 36 that feeds a sterilant to vaporizer 28, flow elements 59, and first and second humidity sensors 16 and 68. Controller 110 also controls the operation of heater 56 and vaporizer 28. Controller 110 is a system microprocessor or a micro-controller that is programmed to control the operation of system 10. Controller 110 controls the flow position of first valve 88 and second valve 92 by providing an electronic signal to first valve 88 and second valve 92. Based on the selected flow position, first valve 88 and second valve 92 control the carrier gas flow rate along first fluid flow path “A” and second fluid flow path “B.” First valve 88 and second valve 92 can increase the flow of the carrier gas along first fluid flow path “A” while decreasing the flow of the carrier gas along second fluid flow path “B” or vice versa. In the preferred embodiment, first valve 88 and second valve 92 are capable of causing more carrier gas to flow along first fluid flow path “A” than second fluid flow path “B” or vice versa. Controller 110 may also control the speed of motor 84 to provide a reduced flow to either flow path.

An input unit 112 is provided and attached to controller 110 to allow a user of system 10 to input operation parameters. Input unit 112 may be any device that would facilitate the input of data and information to controller 110 by a user of system 10, such as by way of example and not limitation, a keypad, a keyboard, a touch screen or switches. An output unit 114 is also connected to controller 110. Output unit 114 is provided to enable controller 110 to provide information to the user on the operation of system 10. Output unit 114 may be, by way of example and not limitation, a printer, display screen or LED display. Controller 110 is programmed such that system 10 operates in certain operating phases while maintaining certain preferable operating conditions.

The present invention shall now be further described with reference to the operation of system 10. A typical sterilization/decontamination cycle includes a drying phase, a conditioning phase, a decontamination phase and an aeration phase. Prior to the initiation of a sterilization/decontamination cycle, input unit 112 is used to provide the operational parameters to controller 110. The operational parameters include a target humidity level for a drying phase, a target VHP concentration for a conditioning phase, a target humidity level and a target VHP concentration for a decontamination phase, and a target VHP concentration for an aeration phase.

When a sterilization/decontamination cycle is first initiated, controller 110 starts with a drying phase. Controller 110 causes motor 84 to drive blower 82, thereby causing a carrier gas to circulate simultaneously along first and second fluid flow paths “A” and “B.” During this phase, controller 110 positions valves 88 and 92 such that the majority of the carrier gas will flow along second fluid flow path “B.” The carrier gas will be directed between the flow paths by controller 110 as required for correct system operation. During the drying phase, vaporizer 28 is not operating, but is being heated to operating temperature. Dryer 64 removes moisture from the air circulating through second fluid flow path “B.” Throughout the drying phase, first humidity sensor 16 provides a signal to controller 110 that is proportional to the actual humidity level of the carrier gas in isolator or room 12. Throughout the drying phase, controller 110 periodically compares the actual humidity level, as measured by first humidity sensor 16, to the target humidity level for the drying phase. If the actual humidity level is higher than the target humidity level, controller 110 continues to operate in the drying phase. Once the actual humidity level is lower than the target humidity level controller 110 ends the drying phase.

Following the drying phase, the conditioning phase is then initiated. Controller 110 causes first valve 88 and second valve 92 to move to a flow position to cause a majority of the carrier gas to flow along first fluid flow path “A.” The speed of motor 84 may be adjusted to provide the required flow along fluid flow path “A”. The carrier gas will be directed between the flow paths by controller 110 as required for correct system operation. Controller 110 activates vaporizer 28 and sterilant supply motor 42 to provide sterilant to vaporizer 28. Within vaporizer 28, the liquid sterilant is vaporized to produce vaporized hydrogen peroxide (VHP) and water vapor, in a conventionally known manner. The vaporized sterilant is introduced into first fluid flow path “A” and is carried by the carrier gas into sterilization/decontamination chamber or region 24 within isolator or room 12. Carrier gas is circulated simultaneously through first fluid flow path “A” and second fluid flow path “B.” Because more carrier gas is circulated along first fluid flow path “A” than second fluid flow path “B,” more VHP will be injected into chamber or region 24 and less will be destroyed by destroyer 62. Throughout the sterilization/decontamination cycle, VHP sensor 26 provides a signal to controller 110 that is proportional to the concentration of the VHP in isolator or room 12. Throughout the conditioning phase, controller 110 periodically compares the actual VHP concentration, as measured by VHP sensor 26, to the target VHP concentration of the conditioning phase. If the actual VHP concentration is lower than the target VHP concentration level, controller 110 continues to operate in the conditioning phase. Once the actual VHP concentration is above the target VHP concentration, controller 110 ends the conditioning phase.

After the conditioning phase is completed, the decontamination phase is initiated. During the decontamination phase, controller 110 receives electrical signals from VHP sensor 26 and first humidity sensor 16 that are proportional to the concentration of VHP and the humidity level in isolator or room 12. Throughout the decontamination phase, controller 110 periodically compares the actual VHP concentration and humidity level to the target VHP concentration and the target humidity level for the conditioning phase. If the actual humidity is above the target humidity or the actual VHP concentration is above the target VHP concentration, controller 110 sends an electronic signal to second valve 92. Second valve 92 then opens to a position to cause more carrier gas to flow along fluid flow path “B.” Controller 110 also sends an electronic signal to motor 84 to increase speed to provide increased air flow. Controller 110 sends an additional signal to motor 42. Motor 42 then turns pump 38 at a slower rate to reduce the amount of liquid sterilant being supplied to vaporizer 28. If the actual VHP concentration or the actual humidity level drops below the target VHP concentration or the target humidity level, controller 110 sends an electronic signal to second valve 92, and motor 84. Second valve 92 moves to a position to reduce the carrier gas flow along second fluid flow path “B,” while motor 84 speed is adjusted to reduce air flow. Controller 110 also sends an electrical signal to motor 42. Motor 42 then turns pump 38 at a faster rate to increase the output of liquid sterilant to vaporizer 28. For the remainder of the decontamination phase, controller 110 continues to control valves 88 and 92, motor 84, and motor 42 based on the actual VHP concentration and humidity levels in isolator or room 12. The decontamination phase ends once the target conditions have been achieved in isolator or room 12 for a predetermined period of time that is sufficient to effect the desired sterilization or decontamination of sterilization/decontamination chamber or region 24, and/or items therein.

After the decontamination phase is completed, the aeration phase is initiated. Controller 110 causes first valve 88 and second valve 92 to move to positions wherein the majority of the carrier gas flowing in system 10 flows along second fluid flow path “B.” The carrier gas will be directed between the flow paths by controller 110 as required for correct system operation. Controller 110 also causes vaporizer 28 and motor 42 to turn off thereby stopping the introduction of VHP into first fluid flow path “A.” The aeration phase is run until the vaporized hydrogen peroxide (VHP) level in isolator or room 12 is below an allowable threshold (about 1 ppm). In this respect, as will be appreciated, blower 82 continues to simultaneously circulate the carrier gas and sterilant through the first fluid flow path “A” and second fluid flow path “B,” thereby causing the last of the vaporized hydrogen peroxide (VHP) to be broken down by catalytic destroyer 62.

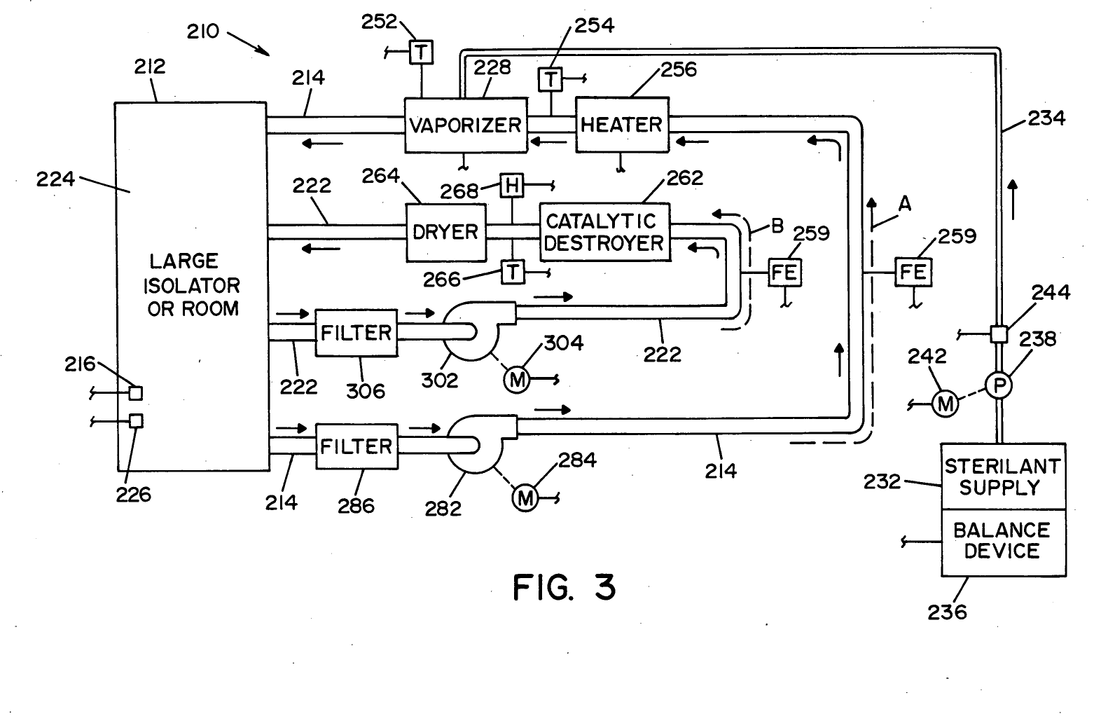

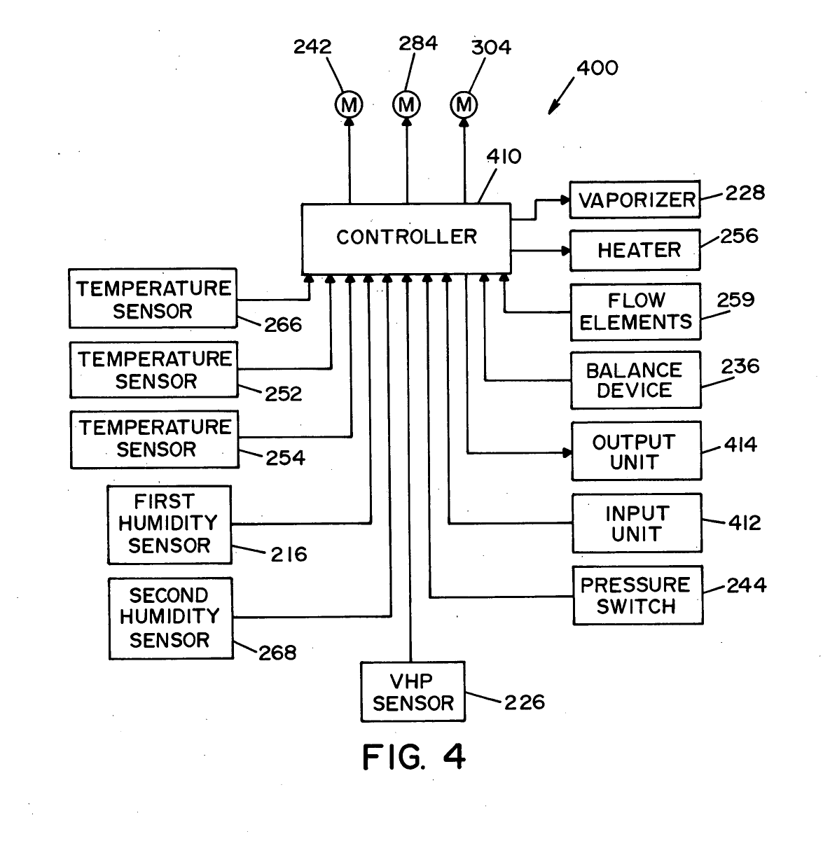

Referring now to FIGS. 3 and 4, an alternative embodiment of a vapor decontamination system 210 is shown. System 210 includes an isolator or room 212 that defines an inner sterilization/decontamination chamber or region 224. Articles to be sterilized or decontaminated, a first humidity sensor 216 and a VHP sensor 226 are disposed within isolator or room 212. Sterilization/decontamination system 210 is comprised of a first fluid flow path “A” and a second fluid flow path “B.” First fluid flow path “A” is defined by isolator or room 212 and a first conduit 214. One end of first conduit 214 connects to isolator or room 212. The other end of a first conduit 214 also connects to isolator or room 212. In this respect, isolator or room 212 and first conduit 214 define a closed loop path. Second fluid flow path “B” is defined by isolator or room 212 and a second conduit 222. One end of second conduit 222 connects to isolator or room 212. The other end of second conduit 222 also connects to isolator or room 212. In this respect, isolator or room 212 and second conduit 222 define a closed loop path.

A vaporizer 228 (also referred to herein as generator) is disposed along first fluid flow path “A” to introduce vaporized hydrogen peroxide into first fluid flow path “A” as described above. A VHP temperature sensor 252, a vaporizer inlet temperature sensor 254 and a heater 256 are also connected to first fluid flow path “A” as described above in the preferred embodiment. As described in the preferred embodiment, a catalytic destroyer 262, a dryer 264, a catalytic destroyer temperature sensor 266, and a second humidity sensor 268 are also provided along second fluid flow path “B.” Other elements such as a pump 238, driven by a motor 242, a balance device 236, a sterilant supply 232, a pressure switch 244 and a feed line 234 are involved in advancing hydrogen peroxide to vaporizer 228. The operation of these elements is described in the preferred embodiment.

A first blower 282, driven by a first motor 284, is provided to circulate a carrier gas along first fluid flow path “A.” A first filter 286 is provided upstream of first blower 282. First filter 286 is operable to filter dirt and/or debris from the carrier gas circulated along first fluid flow path “A.” A flow element 259 is provided in conduit 214 to provide a variable electrical signal that is proportional to the air flow.

A second blower 302, driven by a second motor 304, is provided to circulate a carrier gas along second fluid flow path “B.” A second filter 306 is provided upstream of second blower 302. Second filter 306 is operable to filter dirt and/or debris from the carrier gas circulated along second fluid flow path “B.” A flow element 259 is provided in conduit 222 to provide a variable electrical signal that is proportional to the air flow.

Referring now to FIG. 4, a control system 400 for controlling the operation of system 210 is schematically illustrated. Control system 400 includes a controller 410 that is provided to control the operation of motors 242, 284 and 304. Controller 410 also monitors VHP sensor 226, pressure switch 244, VHP temperature sensor 252, vaporizer inlet air temperature sensor 254, catalytic destroyer air temperature sensor 266, balance device 236 that feeds a sterilant to vaporizer 228, flow elements 259, and first and second humidity sensors 216 and 268. Controller 410 also controls the operation of heater 256 and vaporizer 228. Controller 410 is a system microprocessor or a micro-controller that is programmed to control the operation of system 210. Controller 410 controls the rotational speed of second motor 304 by providing an electrical signal to second motor 304. Second motor 304 then controls the speed of second blower 302. Second blower 302 in turn controls the flow rate of the carrier gas through fluid flow path “B.” Controller 410 operates second motor 304 in response to electrical signals received from VHP sensor 226 and first humidity sensor 216. Controller 410 monitors the actual VHP concentration and humidity level and calculates the appropriate speed of motors 304 and 242 to achieve the target operational conditions. Motor 284 is preferably a single speed motor sized for the desired flow through fluid flow path “A,” but may be speed controlled as illustrated above. It can be appreciated that input unit 412 and output unit 414 are the same as described above in the preferred embodiment.

The alternative embodiment shall now be further described with reference to the operation of system 210. A typical sterilization/decontamination cycle includes a drying phase, a conditioning phase, a decontamination phase and an aeration phase. Prior to the initiation of a sterilization/decontamination cycle, input unit 412 is used to provide the operational parameters to controller 410. The operational parameters include a target humidity level for a drying phase, a target VHP concentration for a conditioning phase, a target humidity level and a target VHP concentration for a decontamination phase, and a target VHP concentration for an aeration phase.

When a sterilization/decontamination cycle is first initiated, controller 410 starts with a drying phase. Controller 410 causes first and second motors 284 and 304 to drive first and second blowers 282 and 302, thereby causing a carrier gas to circulate through system 210 simultaneously along first fluid flow path “A” and second fluid flow path “B.” Preferably, both blowers will be operated at their maximum speed. Dryer 264 removes moisture from the carrier gas circulating through second fluid flow path “B.” Controller 410 ends the drying phase when the actual humidity level, as measured by first humidity sensor 216, is less than the target humidity level.

The conditioning phase is then initiated. Controller 410 continues to cause first and second motors 284 and 304 to drive first and second blowers 282 and 302, thereby causing the carrier gas to simultaneously circulate along first and second fluid flow paths “A” and “B.” It can be appreciated that the remainder of this conditioning phase is operated in the same manner as described in the preferred embodiment in regards to vaporizer 228.

After the conditioning phase is completed, the decontamination phase is initiated. Throughout the sterilization/decontamination cycle, VHP sensor 226 and first humidity sensor 216 send an electric signal to controller 410 that is proportional to the actual VHP concentration and humidity level in isolator or room 212. If the actual VHP concentration or humidity level increases above the target VHP concentration or humidity level, controller 410 causes second motor 304 to drive second blower 302 faster to increase the flow of the carrier gas along fluid flow path “B.” Controller 410 also sends an additional signal to motor 242 to reduce the amount of liquid sterilant being supplied to vaporizer 228. If the actual VHP concentration or humidity level drops below the target VHP concentration or humidity level, controller 410 sends an electronic signal to motors 304 and 242. Motor 304 reduces the flow of the carrier gas along fluid flow path “B,” and motor 242 increases the output of liquid sterilant to vaporizer 228. For the remainder of the decontamination phase, controller 410 continues to control motors 304 and 242 based on the actual VHP concentration and humidity levels in isolator or room 212. The decontamination phase ends once the target conditions have been achieved in isolator or room 212 for a predetermined period of time that is sufficient to effect the desired sterilization or decontamination of sterilization/decontamination chamber or region 224, and/or items therein.

After the decontamination phase is completed, the aeration phase is run to bring the vaporized hydrogen peroxide (VHP) level down to an allowable threshold (about 1 ppm). In this respect, first and second blowers 282 and 302 continue to circulate the carrier gas through the first and second fluid flow paths “A” and “B,” thereby causing the last of the vaporized hydrogen peroxide (VHP) to be broken down by catalytic destroyer 262. During the aeration phase, the flow along second fluid flow path “B” is greater than the flow along first fluid flow path “A.” Controller 410 also causes vaporizer 228 and motor 242 to turn off thereby stopping the introduction of VHP into first fluid flow path “A.”

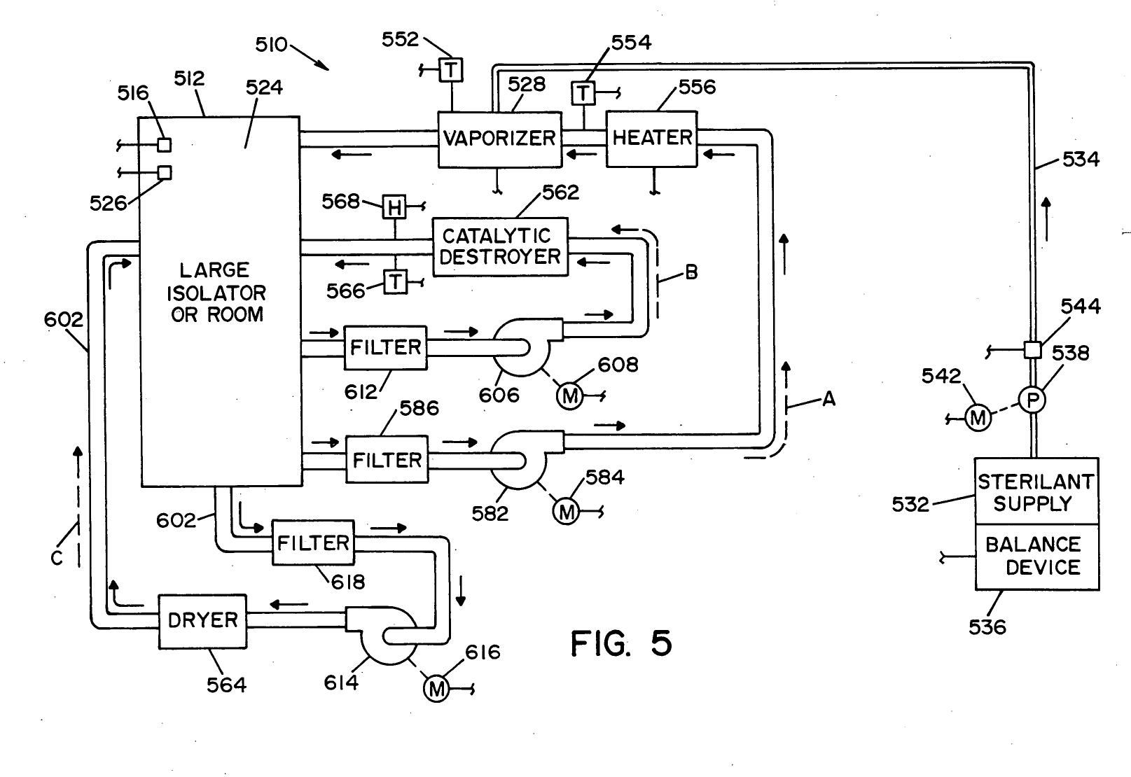

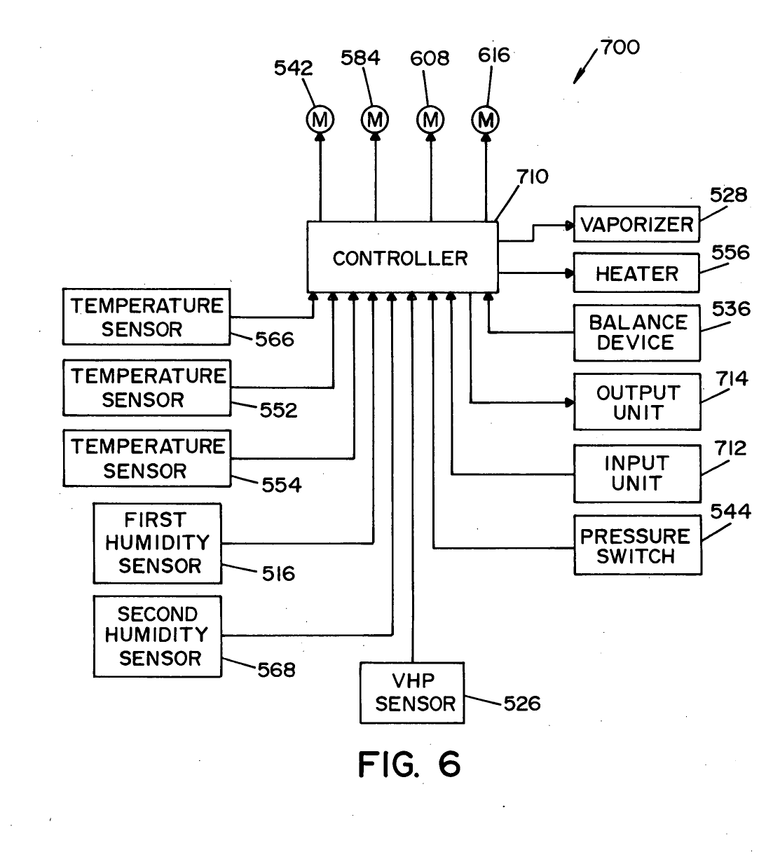

Referring now to FIGS. 5 and 6, another alternative embodiment of a vapor decontamination system 510 is shown. System 510 includes an isolator or room 512 that defines an inner sterilization/decontamination chamber or region 524. A first humidity sensor 516 and a VHP sensor 526 are disposed within isolator or room 512. Sterilization/decontamination system 510 is comprised of a first fluid flow path “A,” a second fluid flow path “B” and a third fluid flow path “C.” First fluid flow path “A” in system 510 is identical to first fluid flow path “A” as defined in system 210. Second fluid flow path “B” in system 510 is identical to second fluid flow path “B” in system 210, except regarding a dryer 564. Third fluid flow path “C” is defined by isolator or room 512 and a third conduit 602. One end of third conduit 602 connects to isolator or room 512. The other end of third conduit 602 also connects to isolator or room 512. In this respect, isolator or room 512 and third conduit 602 define a closed loop path.

First fluid flow path “A” in system 510 includes the same components, a vaporizer 528, a VHP temperature sensor 552, a vaporizer inlet temperature sensor 554 and a heater 556 as described in the preferred embodiment. Other elements such as a pump 538 driven by a motor 542, a balance device 536, a sterilant supply 532, a pressure switch 544 and a feed line 534 are involved in advancing hydrogen peroxide to vaporizer 528. The operation of these elements is described in the preferred embodiment. Second fluid flow path “B” includes a catalytic destroyer 562, a catalytic destroyer temperature sensor 566, and a second humidity sensor 568 as described in the preferred embodiment. Second flow path “B,” however, does not include a dryer 564 as in system 210 above.

Dryer 564 is disposed within third fluid flow path “C.” Dryer 564 is operable to remove moisture from the carrier gas flowing through third fluid flow path

A first blower 582, driven by a first motor 584, is provided to circulate a carrier gas along first fluid flow path “A.” A first filter 586 is provided upstream of first blower 582. First filter 586 is operable to filter dirt and/or debris from the carrier gas circulated along first fluid flow path “A.”

A second blower 606, driven by a second motor 608, is provided to circulate a carrier gas along second fluid flow path “B.” A second filter 612 is provided upstream of second blower 606. Second filter 612 is operable to filter dirt and/or debris from the carrier gas circulated along second fluid flow path “B.”

A third blower 614, driven by a third motor 616, is provided to circulate a carrier gas along third fluid flow path “C.” A third filter 618 is provided upstream of third blower 614. Third filter 616 is operable to filter dirt and/or debris from the carrier gas circulated along third fluid flow path “C.”

A control system 700 for controlling the operation of system 510 is schematically illustrated in FIG. 6. Control system 700 is identical to control system 400 described above, except for the additional control by a controller 710 of third motor 616. Controller 710 sends electronic signals to second and third motors 608 and 616 to operate at various speeds. Second and third motors 608 and 616 then cause second and third blowers 606 and 614 to circulate the carrier gas at various flow rates. It can be appreciated that input unit 712 and output unit 714 are the same as described above in the preferred embodiment.

Alternative embodiment shall now be further described with reference to the operation of system 510. A typical sterilization/decontamination cycle includes a drying phase, a conditioning phase, a decontamination phase and an aeration phase. Depending on the operating phase, the flow along first fluid flow path “A,” or second fluid flow path “B,” or third fluid flow path “C” may be greater than the flow along the other two fluid flow paths. Prior to the initiation of a sterilization/decontamination cycle, input unit 712 is used to provide the operational parameters to controller 710. The operational parameters include a target humidity level for a drying phase, a target VHP concentration for a conditioning phase, a target humidity level and a target VHP concentration for a decontamination phase, and a target VHP concentration for an aeration phase.

When a sterilization/decontamination cycle is first initiated, controller 710 starts with the drying phase. Controller 710 causes first and third motors 584 and 616 to drive first and third blowers 582 and 614, thereby causing a carrier gas to circulate through system 510 along first and third fluid flow paths “A” and “C.” The majority of the carrier gas will flow along third fluid flow path “C.” The carrier gas will be directed between the flow paths by controller 710 as required for correct system operation. Dryer 564 removes moisture from the carrier gas circulating through third fluid flow path “C.” Controller 710 ends the drying phase when the actual humidity level, as measured by first humidity sensor 516, is less than the target humidity level.

During the conditioning phase, controller 710 continues to cause first and third motors 584 and 616 to drive first and third blowers 582 and 614 thereby causing carrier gas to circulate along first and third fluid flow paths “A” and “C.” The remainder of this conditioning phase is operated in the same manner as described in the preferred embodiment in regards to vaporizer 528.

Throughout the sterilization/decontamination cycle, VHP sensor 526 and first humidity sensor 516 send an electronic signal to controller 710 that is proportional to the actual VHP concentration and humidity level in isolator or room 512. Controller 710 periodically compares the actual VHP concentration and humidity levels to the target VHP concentration and humidity levels. If the actual VHP concentration or actual humidity level is higher than the target VHP concentration or humidity level, controller 710 cause third motor 616 to drive third blower 614 faster to provide more flow along fluid flow path “C.” Controller 710 sends an additional signal to motor 542 to reduce the amount of liquid sterilant being supplied to vaporizer 528. If the actual VHP concentration or humidity level drops below the target VHP concentration or humidity level, controller 710 causes third motor 616 to drive third blower 614 slower to provide less flow along third fluid flow path “C.” Controller 710 sends an additional signal to motor 542 to increase the amount of liquid sterilant being supplied to vaporizer 528. For the remainder of the decontamination phase, controller 710 continues to control motors 542, 584 and 616 based on the actual VHP concentration and humidity levels in isolator or room 512. The decontamination phase ends once the target conditions have been achieved in isolator or room 512 for a predetermined period of time that is sufficient to effect the desired sterilization or decontamination of sterilization/decontamination chamber or region 524, and/or items therein.

After the decontamination phase is completed, the aeration phase is run to bring the vaporized hydrogen peroxide (VHP) level down to an allowable threshold (about 1 ppm). In this respect, controller 710 sends a signal to first, second and third motors 584, 608 and 616 causing first, second and third blowers 582, 606 and 614 to simultaneously circulate the carrier gas through the first, second and third fluid flow paths “A,” “B” and “C.” The majority of the carrier gas will flow along second fluid flow path “B” and third fluid flow path “C.” The carrier gas will be directed between the flow paths by controller 710 as required for correct system operation. The vaporized hydrogen peroxide (VHP) flowing along second fluid flow path “B” is broken down by catalytic destroyer 562. Dryer 564 removes moisture of the carrier gas flowing through third fluid flow path “C.”

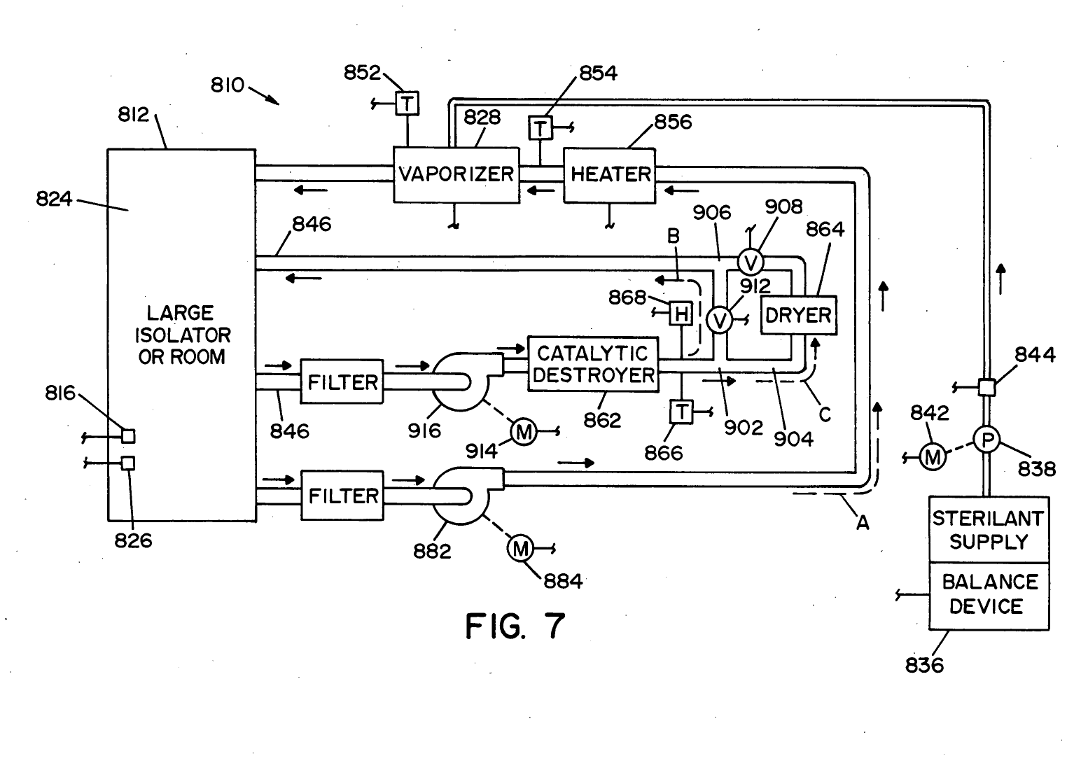

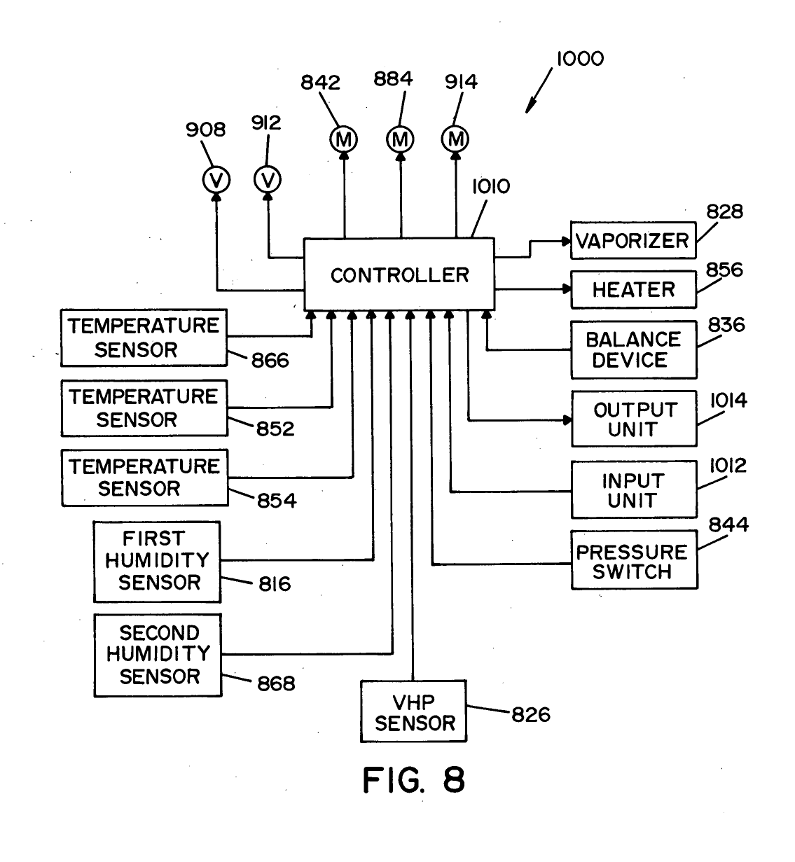

Referring to FIGS. 7 and 8, still another alternative embodiment of a vapor decontamination system 810 is shown. System 810 includes an isolator or room 812 that defines an inner sterilization/decontamination chamber or region 824. A first humidity sensor 816 and a VHP sensor 826 are disposed within isolator or room 812. Sterilization/decontamination system 810 is comprised of a first fluid flow path “A,” a second fluid flow path “B,” and a third fluid flow path “C.” First fluid flow path “A” in system 810 and second fluid flow path “B” are identical to the first and second fluid flow paths “A” and “B” as defined in system 510. Third fluid flow path “C” is defined by isolator or room 812, a portion of a second conduit 846 and a bypass conduit 904. One end of bypass conduit 904 connects to second conduit 846 at a first intersection 902. The other end of bypass conduit 904 connects to second conduit 846 at a second intersection 906. First intersection 902 is located upstream of second location 906. In this respect, isolator or room 812, a portion of second conduit 846 and bypass conduit 904 define a closed loop path.

A dryer 864 is disposed within bypass conduit 904. Dryer 864 is operable to remove moisture from the carrier gas flowing through third fluid flow path “C.” A dryer valve 908 is disposed between dryer 864 and second intersection 906. Dryer valve 908 is downstream of dryer 864. Dryer valve 908 is operable to control the flow of the carrier gas through third fluid flow path “C.” A bypass valve 912 is located in second fluid flow path “B” downstream of first intersection 902. Bypass valve 912 is located between first intersection 902 and second intersection 906. Bypass valve 912 is a control valve operable to control the flow of the carrier gas along second fluid flow path “B.”

Control system 1000 for controlling the operation of system 810 is schematically illustrated in FIG. 8. Control system 1000 includes a controller 1010 that is provided to control operations of motors 842, 884 and 914. Controller 1010 also monitors VHP sensor 826, pressure switch 844, VHP temperature sensor 852, vaporizer inlet air temperature sensor 854, catalytic destroyer air temperature sensor 866, balance device 836 that feeds a sterilant to vaporizer 828, and first and second humidity sensors 816 and 868. Controller 1010 also controls the operation of heater 856 and vaporizer 828. Controller 1010 is a system microprocessor or a micro-controller that is programmed to control the operation of system 810. Controller 1010 controls motors 884 and 914. Motors 884 and 914 in turn control the speed of blowers 882 and 916. Blowers 882 and 916 control the rate of flow of the carrier gas along fluid flow paths “A,” “B” and “C.” Controller 1010 provides an electronic signal to valves 908 and 912 to control the position of valves 908 and 912. Valves 908 and 912 control the flow of the carrier gas along fluid flow paths “B” and “C.” It can be appreciated that input unit 1012 and output unit 1014 are the same as described above in the preferred embodiment.

Operation of this embodiment shall now be further described with reference to the operation of system 810. A typical sterilization/decontamination cycle includes a drying phase, a conditioning phase, a decontamination phase and an aeration phase. Depending on the operating phase, the flow along first fluid flow path “A,” or second fluid flow path “B,” or third fluid flow path “C” may be greater than the flow along the other two fluid flow paths. Prior to the initiation of a sterilization/decontamination cycle, input unit 1012 is used to provide the operational parameters to controller 1010. The operational parameters include a target humidity level for a drying phase, a target VHP concentration for a conditioning phase, a target humidity level and a target VHP concentration for a decontamination phase, and a target VHP concentration for an aeration phase.

When a sterilization/decontamination cycle is first initiated, controller 1010 starts with the drying phase. Controller 1010 causes first motor 884 and second motor 914 to drive first blower 882 and second blower 916, thereby circulating the carrier gas along first, second and third fluid flow paths “A,” “B” and “C.” Controller 1010 sends an electrical signal to bypass valve 912 and dryer valve 908. Bypass valve 912 and dryer valve 908 in turn cause an increase in the flow of the carrier gas along third fluid flow path “C” and a decrease in the flow of the carrier gas along second fluid flow path “B.” The majority of the carrier gas will flow along third fluid flow path “C.” The carrier gas will be directed between the flow paths by controller 1010 as required for correct system operation. During the drying phase, first motor 884 and first blower 882 are both operating to circulate the carrier gas along first fluid flow path “A.” Dryer 864 removes moisture from the carrier gas circulating through third fluid flow path “C.” Controller 1010 ends the drying phase when the actual humidity level, as measured by first humidity sensor 816, is less than the target humidity level.

The conditioning phase is then initiated. Controller 1010 causes motor 842 to drive pump 838 to supply hydrogen peroxide to vaporizer 828. It can be appreciated that the remainder of this conditioning phase is operated in the same manner as described in the preferred embodiment with regards to vaporizer 828.

After the conditioning phase is completed, the decontamination phase is initiated. During the decontamination phase, the sterilant injection rate to vaporizer 828 and to sterilization/decontamination chamber or region 824 is decreased to maintain the concentration of vaporized hydrogen peroxide (VHP) constant and at a desired level. Controller 1010 uses VHP sensor 826 and first humidity sensor 816 to continuous monitor the VHP concentration and the humidity level in isolator or room 812. As the humidity level or the VHP concentration exceeds the target VHP concentration or humidity level, controller 1010 sends a signal to bypass valve 912 and dryer valve 908. Bypass valve 912 moves to a position to reduce the flow of the carrier gas along second fluid flow path “B” while dryer valve 908 moves to a position to increase the flow of the carrier gas along third fluid flow path “C.” Controller 1010 also sends an electrical signal to motor 842. Motor 842 reduces the amount of liquid sterilant being supplied to vaporizer 828. As the concentration of VHP and water in the isolator or room 812 drops below the target levels, controller 1010 sends an electronic signal to valves 908 and 912. Bypass valve 912 and dryer valve 908 reduce the carrier gas flow along fluid flow path “C.” Controller 1010 also sends a third signal to motor 842. Motor 842 in turn increases the output of liquid sterilant to vaporizer 828. For the remainder of the decontamination phase, controller 1010 continues to control motors 914, and 842 and valves 908 and 912 based on the actual VHP concentration and humidity levels in isolator or room 812. The decontamination phase ends once the target conditions have been achieved in isolator or room 812 for a predetermined period of time that is sufficient to effect the desired sterilization or decontamination of sterilization/decontamination chamber or region 824, and/or items therein.

After the decontamination phase is completed, controller 1010 causes vaporizer 828 and motor 842 to stop, thereby stopping the supply of vaporized hydrogen peroxide to the carrier gas flowing along first fluid flow path “A.”

Thereafter, the aeration phase is run to bring the vaporized hydrogen peroxide (VHP) level down to an allowable threshold (about 1 ppm). In this respect, as will be appreciated, first blower 882 and second blower 916 continue to circulate the carrier gas and sterilant through the second fluid flow path “B,” thereby causing the last of the vaporized hydrogen peroxide (VHP) to be broken down by catalytic destroyer 862. It can be appreciated that the majority of the carrier gas will flow along second fluid flow path “B” and third fluid flow path “C.” The carrier gas will be directed between the flow paths by controller 1010 as required for correct system operation.

The foregoing descriptions are a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

Having described the invention, the following is claimed:

1. A closed loop vapor decontamination system for decontaminating a defined region, said system comprising:
a chamber defining a region;
a first fluid flow path connected at both ends to said chamber to define a first closed loop path through said chamber;
a second fluid flow path connected at both ends to said chamber to define a second closed loop path through said chamber;
a means for conveying a carrier gas simultaneously along said first and said second fluid flow paths;
a generator for generating vaporized hydrogen peroxide disposed along said first fluid flow path for introducing vaporized hydrogen peroxide into said carrier gas as it circulates through said first fluid flow path;
a destroyer for converting said vaporized hydrogen peroxide into water and oxygen disposed along said second fluid flow path for breaking down said vaporized hydrogen peroxide in said carrier gas as it circulates through said second fluid flow path; and
a controller controlling the amount of said carrier gas flowing along said first and second fluid flow paths.

2. A vapor decontamination system as defined in claim 1, further comprising a heater upstream from said generator in said first fluid flow path, said heater operable to heat said carrier gas flowing along said first fluid flow path.

3. A vapor decontamination system as defined in claim 1, further comprising a dryer downstream from said destroyer in said second fluid flow path, said dryer operable to remove moisture from said carrier gas flowing along said second fluid flow path.

4. A vapor decontamination system as defined in claim 1, further comprising a first valve disposed along said first fluid flow path and a second valve disposed along said second fluid flow path, said controller being operable to control said first and second valves thereby controlling the flow of said carrier gas along said first and second fluid flow paths.

5. A vapor decontamination system as defined in claim 1, wherein first fluid flow path “A” and second fluid flow path “B” share a common return conduit from said chamber.

6. A vapor decontamination system as defined in claim 1, further comprising a first blower disposed along said first fluid flow path, and a second blower disposed along said second fluid flow path, said controller being operable to control said first and second blowers thereby controlling the flow of said carrier gas along said first and second fluid flow paths.

7. A vapor decontamination system as defined in claim 1, further comprising a third fluid flow path connected at both ends to said chamber to define a closed-loop path through said chamber.

8. A vapor decontamination system as defined in claim 7, further comprising a dryer disposed in said third fluid flow path, said dryer operable to remove moisture from said carrier gas flowing along said third fluid flow path.

9. A vapor decontamination system as defined in claim 7, further comprising a third blower disposed in said third fluid flow path.

10. A vapor decontamination system as defined in claim 7, further comprising a third fluid flow path that includes a portion of said second fluid flow path, said third fluid flow path including a bypass conduit, said bypass conduit connected at both ends to said second fluid flow path, a dryer disposed within said third fluid flow path, said dryer operable to remove moisture from said carrier gas flowing along said third fluid flow path and a valve means for controlling the flow of said carrier gas along said third fluid flow path.

11. A vapor decontamination system as defined in claim 1, wherein said controller is programmed to include a drying phase of operation, a conditioning phase of operation, a decontamination phase of operation and an aeration phase of operation.

12. A vapor decontamination system as defined in claim 1, further comprising a sensor disposed within said chamber to monitor the conditions within said chamber and to provide signals indicative of said conditions, said controller varying the relative flow of carrier gas along said first fluid flow path and said second fluid flow path based on signals from said sensor.

13. A method for controlling the humidity level in an isolator or room, comprising the steps of:
providing a sealable region, a first fluid flow path and a second fluid flow path, said first fluid flow path and said second fluid flow path both include said sealable region;
conveying a flow of a carrier gas simultaneously along said first fluid flow path and said second fluid flow path;
introducing vaporized hydrogen peroxide into said carrier gas flowing along said first fluid flow path; and
destroying said vaporized hydrogen peroxide in said carrier gas flowing along said second fluid flow path.

14. A method as defined in claim 13, wherein said carrier gas is air.

15. A method as defined in claim 13, wherein said destroying step includes catalytically decomposing said vaporized hydrogen peroxide into water and oxygen.

16. A method as defined in claim 13, wherein said method further comprises:
providing a third fluid flow path that includes said sealable region;
conveying a flow of said carrier gas along said third fluid flow path; and
drying said carrier gas flowing along said third fluid flow path.

17. A method for controlling the humidity level in an isolator or room, comprising the steps of:
providing a decontamination system having a sealable region, a first fluid flow path and a second fluid flow path that both include said sealable region and a sensor disposed within said sealable region operable to monitor the conditions within said sealable region to provide signals indicative of said conditions;
conveying a flow of a carrier gas simultaneously along said first fluid flow path and said second fluid flow path;
introducing vaporized hydrogen peroxide into said carrier gas flowing along said first fluid flow path;
destroying said vaporized hydrogen peroxide in said carrier gas flowing along said second fluid flow path; and
varying the relative flow of carrier gas along said first fluid flow path and said second fluid flow path based on signals from said sensor.
drying said carrier gas flowing along said third fluid flow path.

18. A method as defined in claim 17, wherein said carrier gas is air.

19. A method as defined in claim 17, where said destroying step includes catalytically decomposing said vaporized hydrogen peroxide into water and oxygen.

20. A method as defined in claim 17, wherein said method further comprises:
providing a third fluid flow path that includes said sealable region; and
conveying a flow of said carrier gas along said third fluid flow path.
